(19) United States
(12) Patent Application Publication
(10) Pub. No.: US 2011/0183140 A1
(43) Pub. Date: Jul. 28, 2011

(54) Method for Polymer Coating and Functionalization of Metal Nanorods

(75) Inventors: John T. Fourkas, Bethesda, MD (US); Linjie Li, Silver Spring, MD (US); Sanghee Nah, Silver Spring, MD (US)

(73) Assignee: UNIVERSITY OF MARYLAND, COLLEGE PARK, College Park, MD (US)

(21) Appl. No.: 13/009,114

(22) Filed: Jan. 19, 2011

Related U.S. Application Data
(60) Provisional application No. 61/297,406, filed on Jan. 22, 2010.

(51) Int. Cl. D02G 3/36; B05D 3/06; B82Y 5/00; B82Y 15/00

(52) U.S. Cl. 428/378; 427/487; 977/762

(57) ABSTRACT

The present invention relates to polymers and their use in coating metal nanorods (especially gold nanorods), and to the coated nanorods compositions. In particular, the invention relates to a process for forming cetyltrimethylammonium bromide (CTAB)-coated gold nanorods and to such coated nanorods that additionally comprise an external cross-linked polymer coating.

### CROSS-REFERENCE TO RELATED APPLICATIONS

This application claims priority to U.S. Patent Application Ser. No. 61/297,406 (filed Jan. 22, 2010; pending), which application is herein incorporated by reference in its entirety.

### BACKGROUND OF THE INVENTION

1. Field of the Invention

The present invention relates to polymers and their use in coating metal nanorods (especially gold nanorods), and to the coated nanorods compositions. In particular, the invention relates to a process for forming cetyltrimethylammonium bromide (CTAB)-coated gold nanorods and to such coated nanorods that additionally comprise an external cross-linked polymer coating.

2. Description of Related Art

Nanorods made of gold or of other metals show considerable promise for a wide range of applications in biomedical areas and other areas (Ray, S. (2010) “Nanotechniques In Proteomics: Current Status, Promises And Challenges,” Biosensors and Bioelectronics 25:2389-2401; Sandar, R. et al. (2009) “Gold Nanoparticles: Past, Present, and Future,” Langmuir 25(24): 13840-13851;

Walkey, C. et al. (2009) Application of semiconductor and metal nanostructures in biology and medicine,” Hematology 2009:701-707). Currently, several therapeutics are approved for use or are in clinical trials (DeJong, W. H. et al. (2008) “Drug Delivery And Nanoparticles: Applications And Hazards,” Int J Nanomed 3:133-149) and it is expected that nanotechnology will be utilized in many more commercial products in the near future (Aillon, K. L. et al. (2009) Effects of Nanomaterial Physicochemical Properties On In Vivo Toxicity,” Advanced Drug Delivery Reviews 61:457 466).

Two problems have emerged with respect to the growing use of nanotechnologies. First, it is well recognized that the physical and chemical properties of materials used at nanoscopic scale can be dramatically different from their macroscopic properties. Thus, there is a concern that nanomaterials may be accompanied by unexpected toxicities and biological interactions (Aillon, K. L. et al. (2009) Effects of Nanomaterial Physicochemical Properties On In Vivo Toxicity,” Advanced Drug Delivery Reviews 61:457 466).

Secondly, metal (and especially gold) nanorods have been found to morph into spherical particles over time, losing many of their valuable physical properties. Such shape changes can be minimized by coating the nanorods with a polymer, such as cetyltrimethylammonium bromide (CTAB), however, such polymers have been shown to be cytotoxic (Isomaa, B. et al. (1976) “The Subacute And Chronic Toxicity Of Cetyltrimethylammonium Bromide (CTAB), A Cationic Surfactant, In The Rat,” Arch. Toxicol. 35(2):91-96). To circumvent this problem, CTAB may be exchanged for other ligands or for uncrosslinked polymer coatings after the nanorods have been synthesized. However, the stability of nanorods having such coatings is often degraded, and the coatings themselves may not have long term stability in vivo.

Accordingly, despite all prior advances, a need remains for metal nanorods that will exhibit greater structural stability over time. The present invention is directed to this and other needs.

### SUMMARY OF THE INVENTION

The present invention relates to polymers and their use in coating metal nanorods (especially gold nanorods), and to the coated nanorods compositions. In particular, the invention relates to a process for forming cetyltrimethylammonium bromide (CTAB)-coated gold nanorods and to such coated nanorods that additionally comprise an external cross-linked polymer coating.

In detail, the invention concerns a metal nanorod comprising an external cross-linked polymer coating. The invention particularly concerns such a metal nanorod, which comprises a cetyltrimethylammonium bromide (CTAB) coating, wherein the CTAB coating is internal to the cross-linked polymer coating.

The invention is directed to the embodiment wherein such nanorods comprise only a single metal, as well as the embodiment wherein such nanorods comprise one, two, three or more metals, which may be a metal mixture or a metal alloy. The invention is directed to the embodiment wherein such rods comprise gold, nickel, palladium, platinum, copper, silver, zinc or cadmium, or any combination thereof.

The invention is directed to the embodiment wherein the external cross-linked polymer coating of such nanorods is a polymer of an acrylate monomer (especially wherein the acrylate monomer is an ethoxylated trimethylolpropane triacrylate). The invention is particularly directed to the embodiment wherein polymerization of the acrylate monomer is achieved via photopolymerization in the presence of sodium 4-[2-(4-morpholino)benzoyl-2-dimethylamino]-butylbenzene sulfonate (MBS).

The invention is also directed to the embodiments wherein a biomolecule (especially wherein the biomolecule is a peptide, nucleic acid molecule, antibody, enzyme, hormone, or molecular label) is conjugated to the external cross-linked polymer coating of any of the above-described nanorods.

The invention is directed to the embodiments any of the above-described nanorods have a diameter or cross-section of between about 5 nm and about 50 nm, an axial length of between about 20 nm and about 200 nm, and an aspect ratio between about 2:5 and about 4:5.

The invention also provides a method for forming a any of the above-described metal nanorods (and especially gold nanorods), the method comprising forming such metal nanorod in the presence of cetyltrimethylammonium bromide (CTAB), a photopolymerization initiator of CTAB polymerization, an acrylate monomer, and a photopolymerization initiator of acrylate polymerization, such that a CTAB coating is formed over the metal of the nanorod, and a cross-linked polymer coating is formed over the CTAB coating. The invention particularly relates to the embodiment wherein the acrylate monomer is an ethoxylated trimethylolpropane triacrylate and/or wherein polymerization of the acrylate monomer is achieved via photopolymerization in the presence of sodium 4-[2-(4-morpholino)benzoyl-2-dimethylamino]-butylbenzene sulfonate (MBS). The invention particularly relates to the embodiment wherein such method further comprises conjugating a biomolecule (e.g., is a peptide, nucleic acid molecule, antibody, enzyme, hormone, or molecular label) to the cross-linked polymer coating. The invention particularly includes the embodiment of such method wherein the nanorods have a diameter or cross-section of between about 5 nm and about 50 nm, an axial length of between about 20 nm and about 200 nm, and an aspect ratio between about 2:5 and about 4:5.

### BRIEF DESCRIPTION OF THE DRAWINGS

FIG. 1 shows a gold nanoparticle coated with a thin polymer layer using MEMAP.

FIG. 2 shows a schematic of a microfluidic system for creating polymer-coated nanorods. Polymerization (shown as a black outline over the nanorods) occurs selectively at the nanorod's surface via MEMAP driven by the laser beam (shown as a column normal to the flow).

### DETAILED DESCRIPTION OF THE INVENTION

The present invention relates to polymers and their use in coating metal nanorods (especially gold nanorods), and to the coated nanorods compositions. In particular, the invention relates to a process for forming cetyltrimethylammonium bromide (CTAB)-coated gold nanorods and to such coated nanorods that additionally comprise an external cross-linked polymer coating (i.e., so as to form a nanorod having a metal rod, a CTAB coating, and an outer cross-linked polymer coating).

As used herein, the term “nanorod” denotes a substantially cylindrical, polygonal composition, being either solid or hollow, and having a diameter or cross-section of between about 5 nm and about 50 nm, more preferably, between about 10 nm and about 50 nm, and more preferably still between about 10 nm and about 40 nm. Most preferably, such nanorods will have an axial length of between about 20 nm and about 200 nm, more preferably, between about 30 and about 200 nm, and more preferably still between about 40 nm and 200 nm. Most preferably, such nanorods will have an aspect ratio (i.e., the ratio of the width of the nanorod to its length) between about 2:5 and 4:5, between about 1:2 and 7:10, or about 3:5. Exemplary nanorods of the present invention have a diameter of between about 13 nm and about 37 nm and a length of between about 40 nm and about 160 nm with aspect ratio of about 3:5.

Although the present invention is exemplified with regard to gold nanorods, it will be understood that the invention is equally applicable to nanorods of other metals, especially nickel, palladium, platinum, copper, silver, zinc or cadmium. The nanorods of the invention may comprise a single metal or may be an alloy (i.e., a solution mixture) or a composite (i.e., non-solution mixture) comprising one, two, three or more additional metals (for example, both gold and silver) (see, Sun, Y. “Silver Nanowires—Unique Templates For Functional Nanostructures,” Nanoscale 2:1626-1642; Wang, H. et al. (2009) “Nucleic Acid Conjugated Nanomaterials for Enhanced Molecular Recognition,” ACS Nano 3(9):2451-2460).

Any of a variety of methods may be used to form the metal nanorods of the present invention. For example, nanorods (particularly in an array form standing in an anodic aluminum oxide (AAO) template) can be electrochemically grown by alternating current electrolysis in an electrolyte with Pt counter electrodes (current electrolysis 50 Hz, 5 V ac; electrolyte 0.01 M HAuCl₄.4H₂O and 0.1 M H₂SO₄ acid) (Zhou, Z. K. et al. (2011) “Tuning Gold Nanorod-Nanoparticle Hybrids into Plasmonic Fano Resonance for Dramatically Enhanced Light Emission and Transmission,” Nano Letters 11:49-55). More preferably, nanorods are prepared using the citrate-reduction method (see, Jang, S. M. et al. (2004) “Adsorption of 4-Biphenylmethanethiolate on Different-Sized Gold Nanoparticle Surfaces,” Langmuir 20:1922-1927; Enüstün, B. V. et al. (1963) “Coagulation of Colloidal Gold,” J. Am. Chem. Soc. 85(21):3317-3328; Turkevich, J. et al. J(1951) “A Study Of The Nucleation And Growth Processes In The Synthesis Of Colloidal Gold,” Discuss. Faraday. Soc. 11: 55-75; Kimling, M. et al. (2006) “Turkevich Method for Gold Nanoparticle Synthesis Revisited,” J. Phys. Chem. B 110(32):15700-15707)

For example, as an initial step, 0.139 g of HAuCl₄ may be dissolved in 250 ml of distilled water. The solution is then brought to a boil, and 20 ml of a 1 wt % sodium citrate solution added under rapid stirring. Boiling is typically continued to induce further reduction (e.g., for about 20 min). The size of nanorods synthesized in this manner can be measured by either UV/visible absorption or transmission electron microscopy (see, Nah, S. et al. (2009) “Field-Enhanced Phenomena of Gold Nanoparticles,” J. Phys. Chem. A 113:4416-4422; Nah, S. et al. (2010) “Metal-Enhanced Multiphoton Absorption Polymerization with Gold Nanowires,” J. Phys. Chem. C 114:7774-7779).

In a preferred embodiment, the nanorods of the present invention are synthesized using cetyltrimethylammonium bromide (CTAB) to coat the nanorods and help preserve their shapes. Without the CTAB on their surfaces, gold nanorods morph into spherical particles over time, losing many of their valuable physical properties. However, CTAB has been shown to be cytotoxic, and therefore cannot be used in the majority of biomedical applications of interest. The development of stable coatings that can be functionalized readily with molecules with targeting, therapeutic, or other purposes would greatly advance the use of metal nanorods in biomedicine.

The present invention particularly relates to the use of metal-enhanced multiphoton absorption polymerization (“MEMAP”) to form such coatings. Multiphoton absorption polymerization (“MAP”) is based on the absorption of two or more photons of light to excite photoinitiator molecules that drive polymerization in a pre-polymer resin (LaFratta, C. N. et al. (2007) “Multiphoton Fabrication,” Angewandte Chemie (Int. Ed.) 46(33):6238-6258; Maruo, S. et al. (2008) “Recent Progress in Multiphoton Fabrication,” Laser Photonics Rev. 2:100-111; Rumi, M. et al. (2008) “Two-Photon Absorbing Materials and Two-Photon-induced Chemistry,” In: PHOTORESPONSIVE POLYMERS I; Springer: Berlin, 2008; Vol. 213; pp 1). The photons used are of too long of a wavelength to be absorbed individually, and so must be absorbed simultaneously. As a result, the absorption probability scales as the light intensity to the power of the number of photons required for excitation. Excitation, and therefore polymerization, can thus be constrained to occur within the focal volume of a tightly focused laser beam. By using an ultrafast laser, which produces short, intense pulses with a low duty cycle, MAP can be accomplished at low average laser power (see, Nah, S. et al. (2009) “Field-Enhanced Phenomena of Gold Nanoparticles,” J. Phys. Chem. A 113:4416-4422; Nah, S. et al. (2010) “Metal-Enhanced Multiphoton Absorption Polymerization with Gold Nanowires,” J. Phys. Chem. C 114:7774-7779).

Because multiphoton absorption is a non-linear optical process, its probability can be increased substantially by field enhancement. In conventional MAP, short laser pulses are used to drive two photon absorption in a pre-polymer resin, causing it to harden in regions of high intensity (generally at the focus of a laser beam that has been focused through a microscope objective). In MEMAP, the proximity of metal nanostructures of appropriate shapes enhances the field of the laser by orders of magnitude, allowing MEMAP to proceed at intensities that are too low to harden the pre-polymer resin in the bulk (e.g., using 890 nm photons) and which would thus normally be below the threshold for causing polymerization.

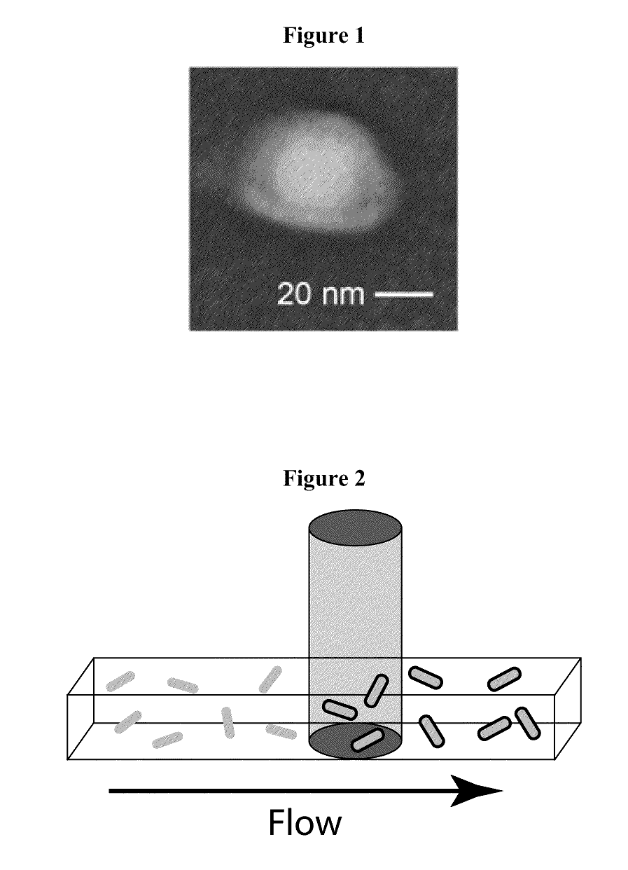

MEMAP generally occurs due to multi-photon absorption-induced luminescence (MAIL) of the metal exciting a photoinitiator (although it could alternatively or additionally occur through enhanced multi-photon absorption of the photoinitiator) (see, Nah, S. et al. (2010) “Metal-Enhanced Multiphoton Absorption Polymerization with Gold Nanowires,” J. Phys. Chem. C 114:7774-7779). However, if the wavelength of the laser is tuned such that two-photon absorption by the photoinitiator is not possible, one can ensure that polymerization occurs only through MAIL and cannot occur at all in the bulk of the pre-polymer resin. Thus, MEMAP provides a means to coat a gold nanostructure selectively with a thin layer of crosslinked polymer. An example of a nanostructure coated in this manner is shown in FIG. 1.

The use of MEMAP has been reported on gold nanostructures created by shadow-sphere lithography (Postnikova, B. J.; (2003) “Towards Nanoscale Three-Dimensional Fabrication Using Two-Photon Initiated Polymerization And Near-Field Excitation,” Microelectron. Eng. 69:459-465), in the controlled gaps of nanoscale gold structures (Sundaramurthy, A. et al. (2006) “Toward Nanometer-Scale Optical Photolithography Utilizing the Near-Field of Bowtie Optical Nanoantennas,” Nano Lett. 6:355-360; Ueno, K. et al. (2008) “Nanoparticle Plasmon-Assisted Two-Photon Polymerization Induced by Incoherent Excitation Source,” J. Am. Chem. Soc. 130(22):6928-6929), and at a metal-coated AFM tip (Yin, X. et al. “Near-Field Multiphoton Nanolithography Using an Apertureless Optical Probe,” In: NONLINEAR OPTICAL TRANSMISSION AND MULTIPHOTON PROCESSES IN ORGANICS). MEMAP has also been observed in arrays of gold nanostructures excited with incoherent light (Ueno, K. et al. (2008) “Nanoparticle Plasmon-Assisted Two-Photon Polymerization Induced by Incoherent Excitation Source,” J. Am. Chem. Soc. 130(22):6928-6929).

The present invention provides a method for coating gold nanorods (or nanorods made of, or containing other metals) with a thin, crosslinked polymer layer that can readily be chemically functionalized. Because this polymer layer is crosslinked, it is less sensitive to degradation in vivo. Moreover, because the CTAB will be encased in the polymer layer, cytotoxic effects are decreased or prevented. The polymer layer can be formed without removing the CTAB from the nanorods, thereby helping to preserve the desired nanotube shape. Functionalization of the polymer layer will make possible the creation of multifunctional nanorods for a wide range of applications. Most preferably, the cross-linking polymer is an acrylic polymer formed, for example by the polymerization (especially through photopolymerization) of an acrylate monomer. Most preferably, such monomer is an ethoxylated trimethylolpropane triacrylate, and a suitable photoinitiator, such as sodium 4-[2-(4-morpholino)benzoyl-2-dimethylamino]-butylbenzene sulfonate (MBS), will be used to achieve polymerization.

In accordance with the present invention MEMAP is preferably performed as disclosed by Nah, S. et al. (2009) “Field-Enhanced Phenomena of Gold Nanoparticles,” J. Phys. Chem. A 113:4416-4422 or Nah, S. et al. (2010) “Metal-Enhanced Multiphoton Absorption Polymerization with Gold Nanowires,” J. Phys. Chem. C 114:7774-7779). In brief, a tunable Ti:sapphire laser (Coherent Mira 900-F™) is preferably employed to produced pulses of, for example, 150 fs duration at a repetition rate of 76 MHz. The beam is preferably introduced into an inverted microscope (e.g., a Zeiss Axiovert 100 ™) through the reflected light-source port and directed to the objective via a dichroic mirror. A 1.45 NA, 100×, oil-immersion objective (Zeiss R Plan-FLUAR™) may be used for imaging and multiphoton fabrication. Scanning may be performed with a piezoelectric sample stage or with a set of galvanometric mirrors. The luminescence signal may be collected via a single-photon-counting avalanche photodiode (EG&G) and the signal may be transferred to a computer with use of, for example, a data acquisition board of National Instruments. Data collection and image construction may be performed with software written in LabView™ (National Instruments). An area of 30 μm² may typically be scanned with 140×140 pixel resolution in approximately 10 s. Filters that cut off the excitation light may be placed in front of the detector. Wavelengths ranging from 725 to 809 nm are preferred.

Suitable photoinitiators for CTAB polymerization include Lucirin TPO-L™, which has a two-photon polymerization action spectrum that has a peak at 725 nm, and which in MAP exhibits negligibly small polymerization action at photon wavelengths longer than 850 nm, as well as the radical photoinitiators, 2-benzyl-2-dimethylamino-1-(4-morpholinophenyl)-butanone-1 (Irgacure 369™, Ciba) or 1-hydroxy cyclohexylphenylketone (Irgacure 184™, Ciba) and SU8 2000 Series™ resist (MicroChem) (see, Nah, S. et al. (2010) “Metal-Enhanced Multiphoton Absorption Polymerization with Gold Nanowires,” J. Phys. Chem. C 114:7774-7779).

The properties of the polymer layer can be controlled in a number of ways. By selecting a desired pre-polymer resin and by controlling the nature and concentration of the monomers and the photoinitiator, and the exposure wavelength, intensity, and time of treatment, one can prepare nanorods whose final polymer layer has a desired functionality or property (such as degree of crosslinking and thickness). For example, if acrylic monomers are used in the pre-polymer resin, the polymer layer will have unreacted acrylate groups on its surface. These acrylate groups will serve as handles for further chemistry to functionalize the polymer-coated nanorods. For instance, reaction with ethylene diamine will provide reactive amine groups on the polymer surface that may be used for the synthesis and/or attachment of peptides, proteins, nucleic acids, targeting molecules, visualization probes, therapeutic molecules, etc. For example, one or more species of biomolecules (e.g., a nucleic acid molecule, antibody, enzyme, hormone (e.g., insulin), blood factor (e.g., thrombin, Factor VIII, erythropoietin, etc.), molecular label (e.g., a radiolabeled molecule, a fluorescent labeled molecule, a hapten, an antigen, etc.), etc. may be conjugated to such functional groups so as to permit the nanorods to be used in diagnostics, imaging or therapeutics. For example, conjugating an antibody to a tumor antigen onto a nanorod permits the nanorod to be used to image cells that express the tumor antigen. Likewise, attaching an enzyme or blood factor onto a nanorod permits the nanorod to carry such biomolecule throughout the circulation and extends its bioavailability and half-life (see, Ray, S. (2010) “Nanotechniques In Proteomics: Current Status, Promises And Challenges,” Biosensors and Bioelectronics 25:2389-2401.

In one preferred embodiment of this invention, the nanorods will be composed of gold and will have a diameter of between 13 nm and 37 nm and a length of between 40 nm and 160 nm with an aspect ratio of 3 to 5. The pre-polymer resin will be an aqueous solution of a water-soluble monomer such as ethoxylated trimethylolpropane triacrylate and a water-soluble photoinitiator such as sodium 4-[2-(4-morpholino)benzoyl-2-dimethylamino]-butylbenzene sulfonate (MBS).

The solution of nanorods will be flowed through a microfluidic channel, such as that shown in FIG. 2. An ultrafast laser beam will intersect the channel, and will be tuned to a wavelength at which MAP cannot occur through two-photon absorption in the bulk pre-polymer solution. MEMAP will occur whenever a gold nanorod passes through the laser beam, coating the gold nanorod with a polymer layer. The thickness of the polymer layer can be controlled as described above, and the length of the interaction time of a nanorod with the laser beam can be controlled by adjusting the flow rate of the fluid and/or the focal size of the laser beam. Nanorods can be collected downstream from the interaction region or can undergo reaction and functionalization in subsequent regions of a microfluidic device before collection.

The invention thus provides a method of creating a thin, highly crosslinked polymer layer around gold or other metal nanorods. The layer will allow the nanorods to retain their shape yet be functionalized with molecules for imaging, targeting, therapy, etc.

Having now generally described the invention, the same will be more readily understood through reference to the following examples, which are provided by way of illustration and are not intended to be limiting of the present invention unless specified.

All publications and patents mentioned in this specification are herein incorporated by reference to the same extent as if each individual publication or patent application was specifically and individually indicated to be incorporated by reference in its entirety. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A metal nanorod comprising an external cross-linked polymer coating.

2. The metal nanorod of claim 1, which comprises a cetyltrimethylammonium bromide (CTAB) coating, wherein said CTAB coating is internal to said cross-linked polymer coating.

3. The metal nanorod of claim 1, wherein said nanorod comprises only a single metal.

4. The metal nanorod of claim 1, wherein said nanorod is comprises a metal alloy.

5. The metal nanorod of claim 1, wherein said nanorod comprises gold, nickel, palladium, platinum, copper, silver, zinc or cadmium.

6. The metal nanorod of claim 5, wherein said nanorod comprises gold.

7. The metal nanorod of claim 1, wherein said external cross-linked polymer coating is a polymer of an acrylate monomer.

9. The metal nanorod of claim 7, wherein said acrylate monomer is an ethoxylated trimethylolpropane triacrylate.

10. The metal nanorod of claim 9, wherein polymerization of said acrylate monomer is achieved via photopolymerization in the presence of sodium 4-[2-(4-morpholino)benzoyl-2-dimethylamino]-butylbenzene sulfonate (MBS).

11. The metal nanorod of claim 1, wherein a biomolecule is conjugated to said external cross-linked polymer coating.

12. The metal nanorod of claim 1, wherein said biomolecule is a peptide, nucleic acid molecule, antibody, enzyme, hormone, or molecular label.

13. The metal nanorod of claim 1, wherein said nanorod has a diameter or cross-section of between about 5 nm and about 50 nm, an axial length of between about 20 nm and about 200 nm, and an aspect ratio between about 2:5 and about 4:5.

14. A method for forming a metal nanorod, said method comprising forming a metal nanorod in the presence of cetyltrimethylammonium bromide (CTAB), a photopolymerization initiator of CTAB polymerization, an acrylate monomer, and a photopolymerization initiator of acrylate polymerization, such that a CTAB coating is formed over the metal of said nanorod, and a cross-linked polymer coating is formed over said CTAB coating.

15. The method of claim 14, wherein said metal nanorod comprises gold.

16. The method of claim 14, wherein said acrylate monomer is an ethoxylated trimethylolpropane triacrylate.

17. The method of claim 14, wherein polymerization of said acrylate monomer is achieved via photopolymerization in the presence of sodium 4-[2-(4-morpholino)benzoyl-2-dimethylamino]-butylbenzene sulfonate (MBS).

18. The method of claim 14, wherein said method further comprises conjugating a biomolecule to said cross-linked polymer coating.

19. The method of claim 14, wherein said biomolecule is a peptide, nucleic acid molecule, antibody, enzyme, hormone, or molecular label.

20. The method of claim 14, wherein said nanorod has a diameter or cross-section of between about 5 nm and about 50 nm, an axial length of between about 20 nm and about 200 nm, and an aspect ratio between about 2:5 and about 4:5.
